(19) United States
(12) Patent Application Publication
(10) Pub. No.: US 2010/0082096 A1
(43) Pub. Date: Apr. 1, 2010

(54) Tailored Luminal & Abluminal Drug Elution

(75) Inventor: Daniel Gregorich, St. Louis Park, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 12/567,337

(22) Filed: Sep. 25, 2009

Related U.S. Application Data
(60) Provisional application No. 61/101,426, filed on Sep. 30, 2008.

(51) Int. Cl. A61F 2/82

(52) U.S. Cl. 623/1.43; 623/1.42

(57) ABSTRACT

A stent has a plurality of members made from a first material. At least one of the plurality of members defines at least one hole. Each hole extends from a first surface of a member. Each hole has a first opening and a depth. Each hole has a barrier dividing the hole into a first reservoir and a second reservoir. The first reservoir extends from a first side of the barrier to the first opening of the hole and the second reservoir extends from a second side of the barrier to the second opening of the hole. The barrier is made from a material different from the first material. The barrier is semi-permeable and non-biodegradable and has a thickness which is less than the depth of the hole.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

Stents, grafts, stent-grafts, vena cava filters, expandable frameworks, and similar implantable medical devices are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Radially expandable endoprostheses are often used for delivery of a beneficial therapeutic agent, such as a drug, to an organ or tissue in the body over an extended period of time. These devices may deliver therapeutic agents to a wide variety of bodily systems to provide a wide variety of treatments.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R §156(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, the invention is directed to a stent comprising a plurality of members. At least a portion of the members have at least one hole that has a barrier disposed within the hole. In some embodiment, the hole is a through-hole, In other embodiments, the hole is a blind hole. In some embodiments, the barrier is semi-permeable. In other embodiments, the barrier is bio-erodible and impermeable. The barrier divides the hole into two reservoirs, each having a therapeutic agent deposited therein. First the first therapeutic agent elutes from the first reservoir and then, a period of time later, the second therapeutic agent elutes from the second reservoir. The second therapeutic agent either elutes after diffusing through the semi-permeable barrier or, if the barrier is bio-erodible, after the erosion of the barrier.

These and other embodiments which characterize the invention are pointed out with particularity in the claims annexed hereto and forming a part hereof. However, for further understanding of the invention, its advantages and objectives obtained by its use, reference can be made to the drawings which form a further part hereof and the accompanying descriptive matter, in which there is illustrated and described an embodiments of the invention

### BRIEF DESCRIPTION OF THE SEVERAL VIEWS OF THE DRAWING(S)

A detailed description of the invention is hereafter described with specific reference being made to the drawings

FIG. 1 is a perspective view of a stent comprising a plurality of members.

FIG. 2 is a top view of a portion of a member of the stent in FIG. 1.

FIG. 3 is a cross-sectional view of a member with a hole that is in the form of a through hole.

FIG. 4 is a cross-sectional view of a member with two holes, each hole having a barrier positioned within the hole.

FIG. 5 is a cross-sectional view of a member with two holes, each hole having a barrier positioned within the hole.

FIG. 6 is a cross-sectional view of a member with two holes, each hole having a barrier positioned within the hole.

FIG. 7 is a cross-sectional view of a member with a hole that is in the form of a blind hole with a barrier positioned within the hole.

FIG. 8 is a cross-sectional view of a member with two holes, one hole in the form of a through hole and one hole in the form of a blind hole.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

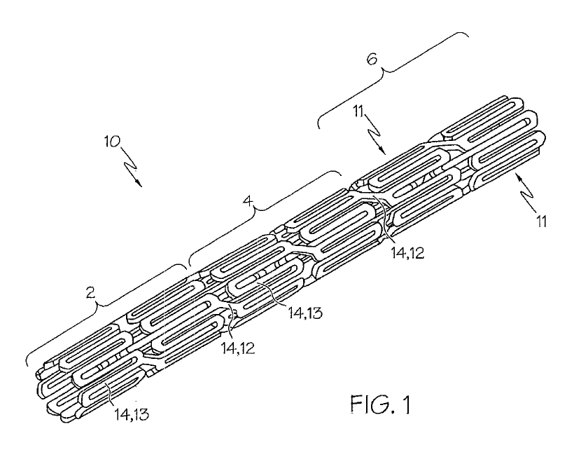

As shown in the figures, the invention is directed to a stent 10 with a plurality of holes 20 where each hole 20 is divided into at least two reservoirs 24 by a barrier 22 and at least one therapeutic agent 26 is deposited into each reservoir 24 so that the stent 10 can deliver at least one therapeutic regimen. The stent 10 can have any configuration, as shown, for example in FIG. 1 which shows a stent 10 that comprises a plurality of members 14 forming circumferential rings 11 about the circumference of the stent 10.

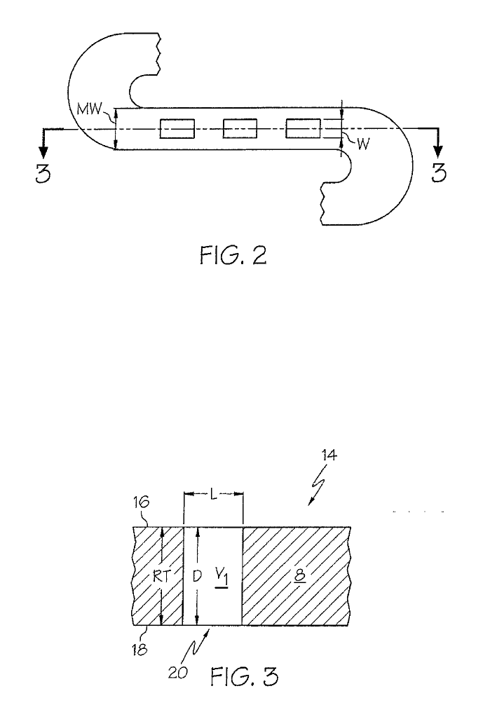

Members 14, as used in this application, include struts 13 and connectors 12. Each member 14 is made from stent material 8. Examples of suitable stent materials 8 are discussed in greater detail below. Each member 14 has four sides: the abluminal side 16 (side of member 14 that is adjacent to the lumen wall), the luminal side 18 (side of member 14 that is adjacent to the lumen) and two sides of the member 14 which are at an oblique angle to the luminal and abluminal sides 16,18 of the member 14 As used in this application, an oblique angle is any angle between 0 and 180 degrees and includes 90 degrees. Each member 14 has a circumferential width (MW) and a radial thickness (RT), as shown in FIGS. 2 and 3.

Holes 20, as used in this application, include through holes 20 and blind holes 20. A through hole 20a has a depth (D) equal to the radial thickness (RT) of the stent 10 while a blind hole 20b has a depth (D) less than the radial thickness (RT) of the stent 10. It is within the scope of the invention for the stent 10 to define a plurality of through holes 20, a plurality of blind holes 20, and any combination thereof. Although a hole 20 can extend between any two surfaces of the member 14, for simplicity, this application will focus on holes 20 that have a first opening in the abluminal surface of the member 14 and that extend through the member 14 towards the luminal surface of the member 14, with through holes 20 having a second opening in the luminal surface of the member 14.

It is within the scope of the invention for a member 14 of the stent 10 to have zero, one, two, three, four, five, six, seven, eight, nine, ten or more holes 20. In some embodiments, a member 14 has at least one through hole 20a, at least one blind hole 20b, and any combination thereof. FIG. 8 shows a member 14 that has a through hole 20a and one blind hole 20b. In some embodiments, the members 14 of a first section of the stent 10 have through holes 20 and the members 14 of a second section of the stent 10 have blind holes 20.

It is within the scope of the invention for the hole 20 to have any shape, for example, but not limited to round shaped, square shaped and rectangular shaped Commonly assigned U.S. Pat. No. 7,135,039 to De Scheerder, entitled Intraluminar Perforated Radially Expandable Drug Delivery Prosthesis and a Method for the Production. Thereof, hereby incorporated by reference in its entirety, shows examples holes that have different shapes. As shown in FIG. 3, the holes 20 have side(s) that are perpendicular to the surfaces 16,18 of the stent 10, however it is within the scope of the invention for the side(s) of the hole 20 to be non-perpendicular to the surface of the stent member 14.

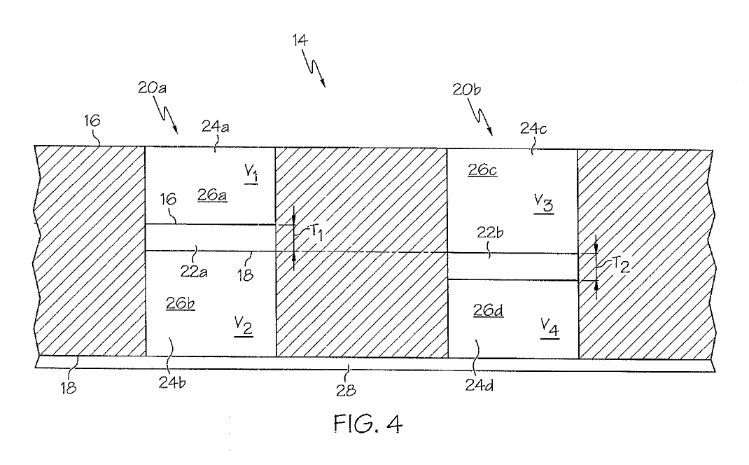

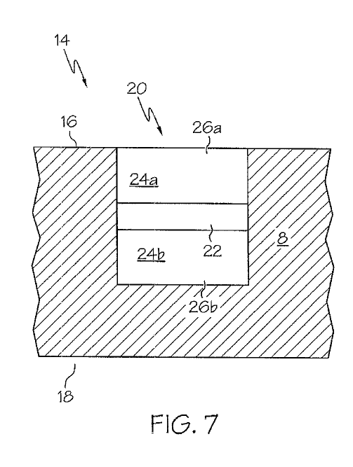

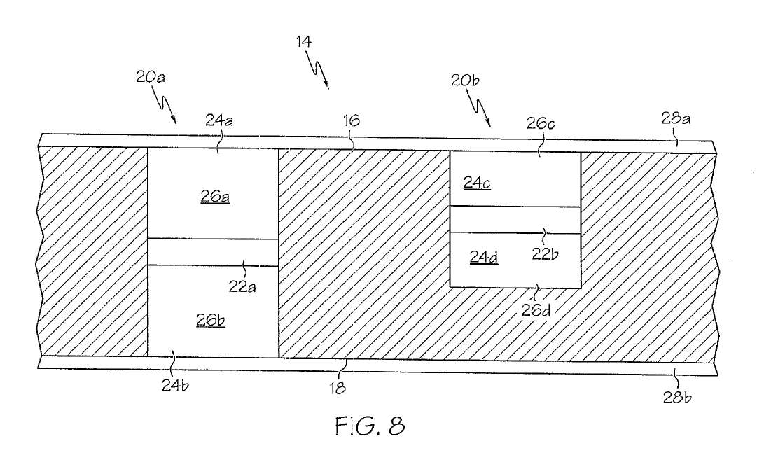

In at least one embodiment, each hole 20 has a barrier 22 positioned within the hole 20 and dividing the hole 20 into two radially adjacent reservoirs 24, as shown, for example, in FIGS. 4 and 7. In some embodiments, each hole 20 has more than one barrier 22 It is within the scope of the invention for a hole 20 to have one, two, three, or more barriers 22. The barrier 22 can be deposited into the hole 20 in any suitable manner, for example, but not limited to, pipetting the barrier material into the hole 20 and depositing the barrier 22 into the hole 20 using an “ink jet” process.

It is within the scope of the invention for the barrier 22 to be permeable, semi-permeable or impermeable. Thus, it is within the scope of the invention for the barrier 22 to have any pore size. In some embodiments, the barrier 22 is biodegradable. In other embodiments, the barrier 22 is non-biodegradable. Examples of materials that can be used to make the barrier 22 are discussed in greater detail below.

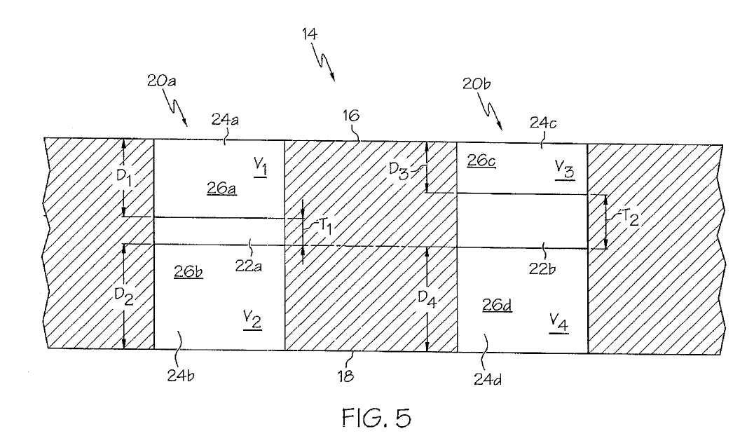

Each barrier 22 has a length, width, and thickness (T). The length of the barrier 22 is equal to the length (L) of the hole 20 and the width of the barrier 22 is equal to the width (W) of the hole 20. It is within the scope of the invention for the barrier 22 to have any thickness (T) that is less than the radial thickness (RT) of the member 14. The barriers 22 in different holes 20 can have the same thickness (T), as shown in FIG. 4. Alternatively, the barriers 22 in different holes 20 can have different thicknesses, as shown, for example, in FIG. 5.

It is within the scope of the invention for the barrier 22 to have any position along the depth of the hole 20. Thus, barriers 22 in adjacent holes 20 can be at the same or different positions, or barriers 22 in different sections of the stent 10 can be at the same or different positions. As shown in FIG. 4, for example, the first barrier 22a has a first position in the first hole 20a and the second barrier 22b has a second position in the adjacent hole 20b, where the first position is different than the second position. One of ordinary skill in the art will recognize that there are many different combinations of barrier positions within and between portions of a stent and all of these different combinations are within the scope of the invention.

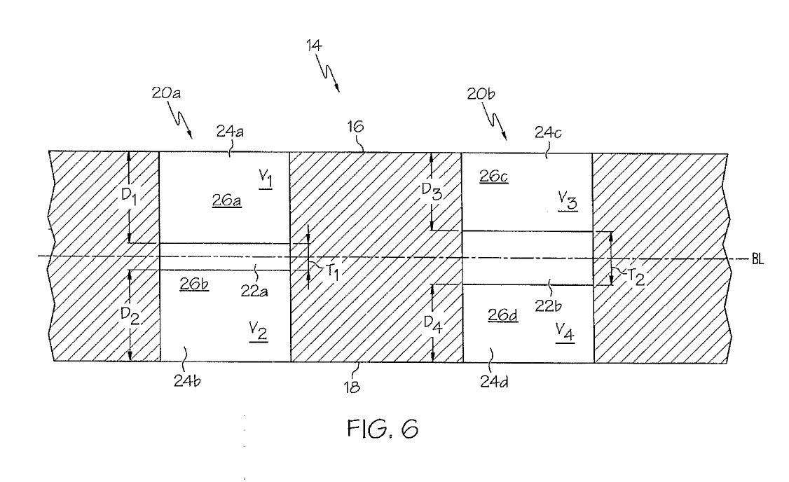

The position of the barrier 22 can be measured by the position of the same surface of the barriers 22, e.g the luminal surface 18, relative to the depth (D) of the hole 20. This is shown, for example, in FIG. 5 where the luminal surfaces 18 of the first and second barriers 22a,b are at the same position, indicated by the dashed line. Alternatively, the position of the barrier 22 can be measured relative to line that bisects the barriers 22 in half; a bisecting line (BL). In FIG. 6, the first and second barriers 22a,b have the same position because the same bisecting line (BL), represented by the dashed line, bisects both barriers 22a,b, even though the first barrier 22a has a different thickness (T₁,T₂) than the second barrier 22b. Thus, the bisecting line (BL) bisects each barrier 22a,b in half.

Each hole 20 has a length (L), width (W), depth (D) and therefore a size/area/volume (V). It is within the scope of the invention for the hole 20 to have any volume (V). In some embodiments, the holes 20 of the stent 10 each have the same volume. In other embodiment, the stent 10 has groups of holes 20 where each group of holes 20 has a different volume. Because the barrier 22 in positioned within the hole 20, the barrier 22 affects the volume (V) of the first and second reservoirs 24a,b. For example, the position and thickness of the barrier 22 affects the volume (V) of the reservoirs 24a,b.

It is within the scope of the invention for the reservoirs 24a,b to have any volume (V). In some embodiments, the first and second reservoirs 24a,b have the same volume (V₁,V₂), as shown, for example in FIG. 5. In other embodiments, the first and second reservoirs 24a,b have different volumes (V₃,V₄), as shown for example in FIG. 5. In at least one embodiment, the first reservoirs 24a of a member 14 are the same volume (V) and the second reservoirs 24b of a member 14 are the same volume (V). In this embodiment, the first and second reservoirs 24a,b can be the same volume or different volumes. Note that in this embodiment, the barriers 22 each have the same position and thickness.

In at least one embodiment, therapeutic agents 26 are deposited into the first and second reservoirs 24 of the holes 20. Note that the volume of therapeutic agent 26 depends upon the size (V) of the reservoir 24. Non-limiting examples of therapeutic agents 26 are discussed in greater detail below. It is within the scope of the invention for the therapeutic agent 26 to be deposited into the reservoirs 24 in any manner.

In some embodiments, the same therapeutic agent 26 is deposited in both the first and second reservoirs 24. It is within the scope of the invention for the therapeutic agents 26 in the first and second reservoirs to have the same concentration or different concentrations. In other embodiments, a first therapeutic agent 26a is deposited into in the first reservoir 24a and a second therapeutic agent 26b is deposited into in the second reservoir 24a where the first and second therapeutic agents 26a,b are different. In one embodiment, the therapeutic agent 26 deposited in the luminal 18 reservoir 24 inhibits cell proliferation and the therapeutic agent 26 deposited in the abluminal 16 reservoir 24 has an anti-thrombosis effect.

After the stent 10 has been implanted into a body cavity, the therapeutic agents 26 are sequentially eluted from the first and second reservoirs 24. The therapeutic agent 26a within the first reservoir 24a is eluted first to the vessel wall and/or any lesions or affected areas thereon. Then a second therapeutic agent 26b within the second reservoir 24b is eluted to the vessel wall and/or any lesions or affected areas thereon. Thus, the stent 10 is able to deliver two rounds of therapeutic agent 26a,b from the hole 20 over a period of time, with the first therapeutic agent 26a being delivered to the vessel wall and/or any lesions or affected areas at an earlier time than the second therapeutic agent 26b, which is delivered to the vessel wall and/or any lesions or affected areas at a later time.

The length of time for the first therapeutic agent 26a to elute from the first reservoir 24a depends in part upon the shape of the reservoir 24a. In some embodiments, the barrier 22 impedes or slows the elution of the second therapeutic agent 26b because the second therapeutic agent 26b has to traverse the barrier 22 before it elutes from the first reservoir 24a to the vessel wall and/or any lesions or affected areas. Thus, the properties of the therapeutic agent 26 and the barrier 22 affect the elution rate of the therapeutic agent 26 through the barrier 22 and therefore affect the elution rate of the therapeutic agent 26 to the vessel wall.

Characteristics of the therapeutic agent 26 that can affect the elution rate, include, but are not limited to, the type of polymer or polymer matrix used to deliver the therapeutic agent 26 and its release profile for the therapeutic agent 26 being delivered; the chain length of the polymer(s) in the polymer matrix; the purity of the therapeutic agent 26; the level and type of crystalinity (polymorphism); morphology of the therapeutic agent 26, e.g, is the agent dispersed in a carrier of phase separated, the size of the particles and their level interconnectivity; the percent of the therapeutic agent 26 relative to the polymer, i.e. the therapeutic agent 26 can be pure (100%) or combined with a polymer carrier and thus less than 100%; the degradability or bio-stability of the therapeutic agent-polymer matrix; the hydrophobicity of the therapeutic agent 26; the thickness of the layer of the therapeutic agent, and any combination thereof

Characteristics of the barrier 22 that can affect the elution rate of the therapeutic agent 26, include, but are not limited to, the hydrophobicity of the barrier 22; the degradability or bio-stability of the battier 22; the permeability of the barrier 22; specific or non-specific interactions between the therapeutic agent 26 and the barrier 22; and any combination thereof. A non-limiting example of a specific interaction between a therapeutic agent 26 and the barrier 22 is the reaction rate between an enzyme bather and a prodrug. An example of a non-specific interaction is the interaction between a hydrophilic therapeutic agent 26 and a hydrophobic barrier 22.

In at least one embodiment, the elution rate of the therapeutic agent 26 through the barrier 22 depends upon the properties of the therapeutic agent 26 and the properties of the barrier 22. For example, if the therapeutic agent 26 is hydrophilic and the barrier 22 is hydrophobic, the elution rate will be slower than if the therapeutic agent 26 and the barrier 22 are both hydrophilic. In some embodiments, the time difference between the elution of the first therapeutic agent 26a and the second therapeutic agent 26b depends upon the permeability of the barrier 22. The permeability of the barrier 22 is affected by the thickness of the barrier 22, and/or the pore size of the battier 22 in relation to the molecule size of the second therapeutic agent 26b. For example, a thicker barrier 22 impedes elution of the second therapeutic agent 26b for a longer period of time than a thinner barrier 22. Similarly, a barrier 22 that has a smaller pore size impedes elution of the second therapeutic agent 26b for a longer period of time than a barrier 22 that has a larger pore size. One of ordinary skill in the art will recognize that the properties of the therapeutic agent 26 and the barrier 22 can be chosen so that a desired elution rate for a therapeutic agent 26 transversing a barrier 22 will be achieved.

In at least one embodiment, a member 14 has at least one cap 28 engaged to a surface of the member 14 defining at least one hole 20. Thus, a member 14 can have one or two caps 28. In this embodiment, the cap 28 extends over the opening(s) of the hole(s) 20 and keeps the therapeutic agent 26 within the reservoir(s) 24 for a period of time, as shown, for example in FIGS. 4 and 8. In some embodiments, a cap 28 is disposed over the (abluminal) opening of a blind hole 20 or over the first (abluminal) opening of a through hole 20. In other embodiments, a cap 28 is disposed over the second (luminal) opening of a through hole 20, as shown, for example, in FIG. 4. In this embodiment, the cap 28 directs the elution of the second therapeutic agent 26b through the barrier 22 and not through the second (luminal) opening of the through hole 20.

In some embodiments, the cap 28 is impermeable and non bio-erodible. In this embodiment, it is within the scope of the invention for the cap 28 to be made of any impermeable and non bio-erodible material that prevents the elution of the second therapeutic agent 26b into the vessel. In other embodiments, the cap 28 is impermeable and bio-erodible. In one embodiment, the material used to make the cap 28 erodes at a slower rate than the material used to make a bio-erodible barrier 22. In another embodiment, the material used to make the cap 28 has an erosion rate that allows the second therapeutic agent 26b to be eluted to both the luminal side and the abluminal side of the member 12. In this embodiment, the cap 28 has an erosion rate that is similar to the erosion rate of the barrier 22. As used in this application, the erosion rate of a material is the rate or speed at which the material erodes. Examples of bio-erodible materials that can be used to make the cap 28 are discussed in greater detail below.

In at least one embodiment, the stents 10 has different regions and/or subregions from which different regimens of therapeutic agents 26 are eluted from the holes 20. As shown, for example in FIG. 1, a stent 10 can be divided into a proximal region 2, a middle region 4 and a distal region 6. In this embodiment each region has two circumferential rings 11 of members 14. One of ordinary skill in the art will recognize that there are numerous ways in which the stent 10 of FIG. 1 can be designed to have different regions ^{and/or} subregions that have different sizes and positions along the longitudinal length of the stent 10. Different regions and/or subregions of a bifurcated stent used to deliver different therapeutic regimens are discussed in greater detail in Bifurcated Stent with Drug Wells for Specific Ostial, Carina, and Side Branch Treatment, Ser. No 11/946,632, with inventors Dan Gregorich, Mike Meyer and Dave Friesen, hereby incorporated by reference herein in its entirety.

As discussed above, the holes 20, the barriers 22, the therapeutic agents 26, the cap 28, and any combination thereof, can be modified and these modifications affect the therapeutic regimen that is eluted from the holes 20. Therefore modification of at least one of these variables in different regions of the stent 10 produces a stent 10 that elutes different regimens of therapeutic agent 26 from different regions of the stent 10. For example, in some embodiments, the reservoirs 24 of members 14 of different regions of the stent 10 are be constructed and arranged as discussed above so that different volumes of first and second therapeutic agents 26 are eluted from different regions of the stent 10 to different areas of the vessel wall. Another example is that the permeability of the barriers 22 in the holes of the first region of the stent 10 is greater than the permeability of the barriers 22 of the second region of the stent 10 so that the second therapeutic agent 26 is eluted from the first region before the second region.

Materials that can be used to make the barrier 22 and/or cap 28 include, but are not limited to, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyanhydride esters, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyorthoesters, polyacrylamides, polyethers, polyether sulfone, polycarbonates, polyiminocarbonates, poly (ester-amides), lysine-containing poly (ester-amides), polyhydroxyalkanoates, 10 poly(propylene fumarate-co-ethylene glycol) copolymers, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid (PLA), poly(glycolic-co-lactic acid) (PLGA), polyglycolic acid (PGA), polycaprolactone, polyhydroxybutyrate (PHB), polyhydroxybutyrate valerate (PHV), poly(hydroxybutyrate-co-valerate (PHBV), and blends and copolymers thereof, as well as other biodegradable, bioabsorbable and biostable polymers and copolymers, polyarylates, free acid polyarylates, polybutylene diglycolate, poly epsilon-caprolactone (PCL), polydihydropyrans, polyphosphazenes, polycyanoacrylates, polyketals, polyacetals, poly(α-hydroxy-esters), a protein polymer, fibrins, collagens, chitins, and derivatives thereof; polysaccharides such as celluloses, cellophane, cellulose nitrate, cellulose propionate, cellulose ethers, carboxymethyl cellulose, starches, dextrans, alginates and derivatives of these polysaccharides, extracellular matrix component, hyaluronic acid, or another biologic agent or a suitable mixture of any of these, silk-elastin polymers, amino acid-containing polymers, recombinant materials such as polydepsipeptides, nylon copolyamides, conventional poly(amino acid) synthetic polymers, pseudo-poly(amino acids), enzymes that convert a prodrug into a therapeutic agent, rubbers, iron, magnesium, corrodible calcium phosphate and magnesium alloys, and any combination thereof. Thus, it is within the scope of the invention for the barrier 22 to be made of one or more materials

The stent material 8 may be any suitable biocompatible materials including one or more polymers, one or more metals or combinations of polymer(s) and metal(s). Examples of suitable stent materials 8 include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The stent material 8 of the inventive stents 10 may be shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The inventive stents 10 may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent 10, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

A therapeutic agent 26 may be a drug or other pharmaceutical product such as non-genetic agents, genetic agents, cellular material, etc. Some examples of suitable non-genetic therapeutic agents 26 include but are not limited to: anti-thrombogenic agents such as heparin, heparin derivatives, vascular cell growth promoters, growth factor inhibitors, Paclitaxel, etc. Where an agent includes a genetic therapeutic agent 26, such a genetic agent may include but is not limited to: DNA, RNA and their respective derivatives and/or components; hedgehog proteins, etc. Where a therapeutic agent 26 includes cellular material, the cellular material may include but is not limited to: cells of human origin and/or non-human origin as well as their respective components and/or derivatives thereof. Where the therapeutic agent 24 includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e. g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A stent, the stent comprising
a plurality of members, each of the plurality of members being made from a first material, at least one of the plurality of members defining at least one hole;
each hole extending from a first surface of the first member to a second surface of the first member and having a depth, each hole having a first opening, each hole having a barrier, the barrier having a position along the depth of the hole, the barrier dividing the hole into a first reservoir and a second reservoir;
the barrier being made from a second material, the second material being a different material than the first material, the second material being semi-permeable and non-biodegradable, the barrier having a thickness which is less than the depth of the at least one hole.

2. The stent of claim 1, further comprising
a first therapeutic agent, the first therapeutic agent being deposited in the first reservoir; and
a second therapeutic agent, the second therapeutic agent being deposited in the second reservoir

3. The stent of claim 2, further comprising a first cap, the first cap being positioned over the first opening of each hole.

4. The stent of claim 3, the at least one hole further comprising a second opening, the stent farther comprising a second cap, the second cap being positioned over the second opening of the hole.

5. The stent of claim 3, the first cap being impermeable.

6. The stent of claim 5, the first cap further being bio-erodible.

7. The stent of claim 6, the first cap eroding at a first rate, the second therapeutic agent eluting at a second rate, the first rate being slower than the second rate.

8. The stent of claim 1, the first therapeutic agent being selected from a group consisting of a cell proliferation inhibitor, an antithrombotic, and an anti-inflammatory;
the second therapeutic agent being selected from the group consisting of a cell proliferation inhibitor, an antithrombotic, and an anti-inflammatory;
the first therapeutic agent being different from the second therapeutic agent

9. The stent of claim 1, the first reservoir having a first size and the second reservoir having a second size, the first size being the same as the second size.

10. The stent of claim 1, the at least one hole comprising a plurality of holes, the barrier in at least one of the plurality of holes having a different position

11. The stent of claim 1, the at least one hole comprising a plurality of holes, the barrier in at least one of the plurality of holes having a different thickness.

12. The stent of claim 1, the at least one hole comprising a plurality of holes, the barrier in at least one of the plurality of holes being more permeable than the barrier in the others of the plurality of holes.

13. The stent of claim 1, the at least one hole comprising a plurality of holes, the first reservoir of at least one of the plurality of holes having a different size.

14. The stent of claim 1, the plurality of members forming a first section and a second section, the plurality of members of the first section defining a plurality of first reservoirs, the plurality of members of the second section defining a plurality of second reservoirs, the first and second reservoirs having the same position relative to the first surfaces of the plurality of members, each of the plurality of first reservoirs having a first size, each of the plurality of second reservoirs having a second size, the first size being larger than the second size.

15. A stent, the stent comprising
a first section and a second section, each section comprising a plurality of members, each of the plurality of members of the first and second sections being made from a first member material and having a thickness,
at least some of the plurality of members of the first section defining at least one hole, each hole having a first barrier, the first barrier being made from a first barrier material, the first barrier material being different than the first member material, the first barrier being positioned along the depth of the hole, the first barrier dividing the hole into a first reservoir and a second reservoir;
at least some of the plurality of members of the second section defining at least one hole, each hole having a second barrier, the second barrier being made from a second barrier material, the second barrier material being different than the first member material, the second barrier being positioned along the depth of the hole, the second barrier dividing the hole into a third reservoir and a fourth reservoir; the third reservoir being smaller than the first reservoir.

16. The stent of claim 15, the at least one hole of the first section being a through hole and the at least one hole of the second section being a through hole.

17. The stent of claim 15, the at least one hole of the first section being a through hole and the at least one hole of the second section being a blind hole

18. The stent of claim 15, the first barrier having a first thickness, the second barrier having a second thickness, the second thickness greater than the first thickness

19. A stent, the stent comprising
a first section and a second section, each section comprising a plurality of members, each of the plurality of members of the first and second sections being made from a first member material and having a thickness,
at least some of the plurality of members of the first section defining at least one hole, each hole having a first barrier, the first barrier being made from a first barrier material, the first barrier material being different than the first member material, the first barrier being positioned along the depth of the hole, the first barrier dividing the hole into a first reservoir and a second reservoir; a first therapeutic agent being deposited in the first reservoir and a second therapeutic agent being deposited in the second reservoir;
at least some of the plurality of members of the second section defining at least one hole, each hole having a second barrier, the second barrier being made from a second barrier material, the second barrier material being different than the first member material, the second barrier being positioned along the depth of the hole, the second barrier dividing the hole into a third reservoir and a fourth reservoir, a third therapeutic agent being deposited in the third reservoir and a fourth therapeutic agent being deposited in the fourth reservoir;
wherein the second therapeutic agent elutes faster than the fourth therapeutic agent.
